(19) United States
(12) Patent Application Publication
(10) Pub. No.: US 2009/0170749 A1
(43) Pub. Date: Jul. 2, 2009

(54) Novel Compounds Which Interact With PEA-15

(75) Inventors: Marcel Hibert, Eschau (FR); Hadjila Chabane, Illkirch (FR); Dominique Bonnet, Geispolsheim (FR); Jacques Haiech, Strasbourg (FR); Francois Renault-Mihara, Tokyo (JP); Hervé M. Chneiweiss, Paris (FR)

Correspondence Address: OLIFF & BERRIDGE, PLC, P.O. BOX 320850, ALEXANDRIA, VA, 22320-4850, US

(73) Assignee: Institut National De La Sante Et De La Recherche Medicale (Inserm), Paris (FR)

(21) Appl. No.: 12/086,591

(22) PCT Filed: Dec. 15, 2006
(86) PCT No.: PCT/FR2006/051367

§ 371 Date: Oct. 16, 2008

(30) Foreign Application Priority Data
Dec. 16, 2005 (FR) 0553921

(51) Int. Cl. A61K 38/04; C07K 2/00; G01N 33/68

(52) U.S. Cl. 514/2; 530/300; 530/331; 435/7.1

(57) ABSTRACT

The present invention relates to novel pseudopeptide compounds of defined formula, capable of interacting with the PEA-15 protein and to the use thereof in screening methods and to a method of diagnosing pathological conditions which may involve PEA-15.

The present invention relates to compounds which may interact with PEA-15 protein (Phosphoprotein Enriched in Astrocytes, with a molecular weight of 15 kDa), their fluorescent derivatives, as well as the implementation of these compounds in methods of screening and diagnosing, and pharmaceutical compositions.

PEA-15 is a small cytoplasmic protein comprising 130 amino acids abundantly expressed in the brain, particularly in astrocytes and to a lesser degree, ubiquitously, in many other tissues.

The structure of this protein, very conserved among the vertebrates, includes at the N-terminus a Death Effector Domain (DED) of 80 amino acids, and a NES domain (Nuclear Export Signal), and a C-terminus of low organized structure containing phosphorylation sites for the protein kinase C (PKC), and for the type II calcium/calmoduline-dependant protein kinase.

The genomic sequence of PEA-15 is made up of four exons and extends over approximately 10.2 kb of genomic DNA (Wolford et al., 2000, Gene, 241:143).

PEA-15 is present in vivo in various forms: non-phosphorylated, mono- and bi-phosphorylated, each one presenting a different biological activity.

PEA-15 is a multifunctional protein which may interact with many partners by means of its various functional domains, and according to its degree of phosphorylation (Renault et al., Biochem. Pharmacol, 2003, 66: 1581). To date, seven partners of PEA-15 have been identified, intervening in the multiple functions fulfilled by this protein, namely FADD, caspase 8, Omi/HtraA2, ERK1/2, Akt, Rsk2, and phospholipase D1. By these multiple interactions, PEA-15 appears to play a central role in many physiological and/or pathological cellular processes.

It has been shown that this protein has, in particular, the properties to inhibit apoptosis, to inhibit the entry of cells in the cell cycle, to be involved in the re-establishment of integrins signaling inhibited by the expression of H-Ras oncogene, to inhibit the cell proliferation, and to be involved in the trans-port of glucose and the secretion of insulin (Renault et al., Biochem. Pharmacol., 2003, 66: 1581).

For example, it has been observed that the suppression of PEA-15 expression in the astrocytes results in an increase in the sensitivity of astrocytes to the apoptosis induced by the TNF alpha (Kitsberg et al., J. Neurosci., 1999, 19: 8244) and that the reduction of the expression of this protein causes an increase in the proliferation of various cell lines such as astrocytes, lymphocytes and hepatocytes (Formstecher et al., Dev. Cell., 2001, 1: 239). It was also observed that the expression of the protein also inhibits cell migration.

In addition, this protein could also be involved in the genesis and/or development of cerebral primitive tumors, as well as in metastatic processes.

For example, an increase in the expression of PEA-15 was observed in various tumors such as gliomas, ovarian cancer, kidney cancer, breast cancer, hepatocellular carcinomas, lymphomas or melanomas (Hwang et al., Genomics, 1997, 42: 540; Bera et al., Proc. Natl. Acad. Sci. USA, 1994, 91: 9789).

Also, the over-expression of this protein in transgenic mice increases their sensitivity to chemically induced skin cancers (Formisano et al., Oncogene, 2005, 24: 7012).

On the other hand, the expression of PEA-15 in a tissue induces an inhibition of the permissiveness of this fatter vis-à-vis the invasion by tumor cells.

It has also been shown that breast cancer cells could be sensitized to chemotherapy by reduction of the expression of PEA-15 (Stassi et al., Cancer Res., 2005, 65: 6668).

WO 2004/108961 proposes the use of PEA-15 as a marker and therapeutic target for papillomas.

Moreover, an over-expression of PEA-15 has been observed in the fibroblasts, the skeletal muscles and the adipose tissue of patients affected by type II diabetes (Condorelli et al., EMBO. J., 1997, 17: 3858), and it was shown that the suppression of the expression of this protein made it possible to restore insulin secretion in response to glucose.

An over-expression of this protein is also observed in certain inflammatory processes.

EP 1,189,060 proposes the use of PEA-15 as a marker and therapeutic target in neurodegenerative diseases.

Consequently, it appears that PEA-15 could be used as a therapeutic target in many pathological conditions. However, to date, there is no easily accessible compounds which may modulate the activity of this protein.

In addition, there is also no tools which may allow easy screening of such compounds.

Consequently, there is a need to easily access compounds which may interact with PEA-15 and modulate its activity.

There is also a need to have screening tools of compounds which may modulate the biological activity of PEA-15.

There is also a need to have novel compounds for the treatment of a pathological condition such as cancer and type II diabetes.

There is also a need to have tools for the diagnosis and/or the prognosis of pathological conditions involving PEA-15, and notably an alteration of its expression and even of its biological activity, such as type II diabetes, cancer, notably gliomas, carcinomas, or pathological conditions involving an excess or a default in apoptosis or cell proliferation, for example.

The object of the present invention is to give satisfaction to these needs.

In an unexpected way, the inventors have observed that the compounds of the following general formula (I):

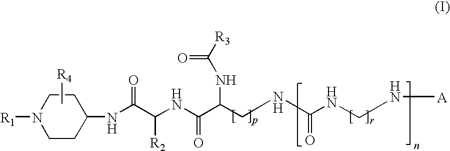

wherein n, p, r, R₁, R₂, R₃, R₄ and A are as defined hereafter, may interact with PEA-15 and modulate its activity.

Thus, the inventors have observed that a compound according to the invention, such as the fluorescent compound 6D6-1 for example, detailed hereafter, is able to interact in a specific way with a PEA-15 protein and to modulate its biological activity.

In addition, the inventors have also observed that it was possible to employ a compound of the invention, notably fluorescent, in combination with a fluorescent fusion protein GFP-PEA-15 (Green Fluorescent Protein) to develop methods implementing a fluorescence resonance energy transfer (FRET) allowing, for example, the screening of agents which may interact with PEA-15 as well as the diagnosis and/or the prognosis of pathological conditions involving PEA-15.

It is also possible to employ such a compound, notably fluorescent, in combination with a fusion protein GST-PEA 15 to implement a method of screening by competition, making it possible to identify agents which may interact with PEA-15.

Thus, according to one of its first aspects, the present invention refers to a compound of the following formula (I):

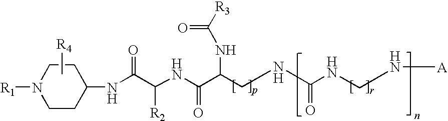

wherein:
n may be equal to 0 or 1,
p may represent an integer varying from 1 to 6, and particularly varying from 2 to 4,
r may represent an integer varying from 1 to 12, particularly varying from 2 to 6, and in particular is equal to 4,
R₁ may represent a hydrogen atom, a saturated or unsaturated, linear or branched C₁-C₂₀ alkyl radical, a saturated or unsaturated C₃-C₁₀ cycloalkyl radical, a C₆-C₁₀ aryl radical, optionally substituted with one or more halogen atom(s), one or more C₁-C₆ alkoxy radical(s), or one or more C₁-C₁₀ alkyl radical(s),
R₂ may represent an amino acid side chain or an amino acid derivative,
—COR₃ may represent an acyl radical carrier of a basic entity R₃, notably selected from the group consisting of radicals with the following formulas:

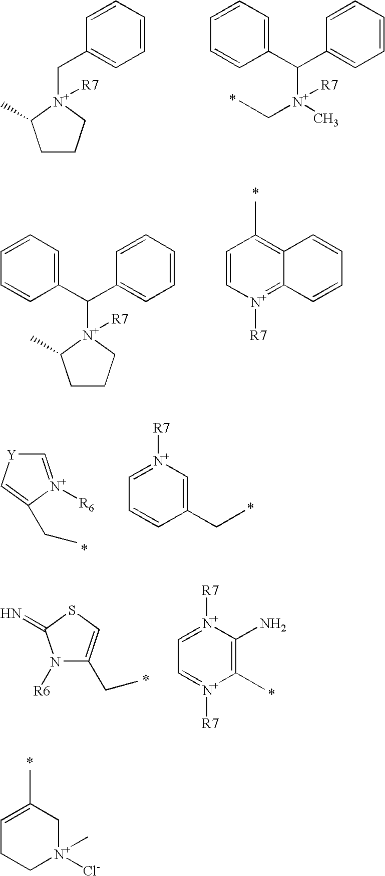

wherein * represents a covalent bond with the acyl radical, Y may represent N or N⁺R₇ and R₆ and R₇ may represent, independently from each other, a hydrogen atom, a saturated or unsaturated, linear or branched C₁-C₂₀ alkyl radical, a saturated or unsaturated C₃-C₁₀ cycloalkyl, a C₆-C₁₀ aryl radical, optionally substituted with one or more halogen atom(s), one or more C₁-C₆ alkoxy radical (s), or one or more C₁-C₁₀ alkyl radical (s),
R₄ may represent a hydrogen atom, a saturated or unsaturated, linear or branched C₁-C₁₀ alkyl radical, a saturated or unsaturated C₃-C₁₀ cycloalkyl radical, a C₆-C₁₀ aryl radical, optionally substituted with one or more halogen atom(s), one or more C₁-C₆ alkoxy radical(s), or one or more C₁-C₁₀ alkyl radicals,
A may represent a radical derived from a xanthene residue, in particular a 9-phenyl xanthene residue, an acridine residue, in particular a 9-phenyl acridine residue or a 4-bora-3a,4a-diaza indacene residue,
and its derivatives.

According to the invention, “residue” in relation to a given molecule, intends to mean the molecule in the form of a radical.

Advantageously, A may represent a fluorescent marker.

According to another of its aspects, the pre-sent invention refers to a screening method of an agent liable to interact with a PEA-15 protein, or an analogue thereof, comprising at least the steps consisting of:
(a) placing at least one PEA-15 protein carrying a fluorescent marker D, or an analogue thereof, in presence of at least one compound, in particular fluorescent, according to the invention, in conditions suitable for an interaction with said protein,
(b) A and D being such that they define a fluorescent energy acceptor-donor pair, suitable for the implementation of a fluorescence resonance energy transfer,
(c) measuring a first signal S₁, characteristic of the assembly obtained in step a) by irradiation at a wavelength, enabling the fluorescent energy donor to be excited,
(d) placing the assembly obtained in step a) in presence of a medium presumed to contain at least one agent to be screened in conditions suitable for an interaction with said protein,
(e) measuring a second signal S₂, of the same type as S₁, characteristic of the assembly obtained in step c) by irradiation at a wavelength enabling the fluorescent energy donor to be excited,
(f) comparing the first and second signals S₁ and S₂ in order to draw a conclusion relating to a possible interaction of said PEA-15 protein with the agent to be screened.

According to the present invention, “PEA-15 protein analogue” intends to mean a peptide compound, presenting a homology of sequences with PEA-15 and a similar biological activity, as well as variants which may result from the alternative splicing of mRNA coding for this protein, such as the one described by Underhill et al. (Mamm. Genome, 2001, 12: 172) for example, as well as fragments of this protein or these peptide compounds type, with the capacity to bind a compound of the formula according to the invention. “Biological activity” intends to mean the biological properties of the PEA-15 protein, notably as previously indicated.

“Homology of sequences” intends to mean a sequence identity of at least 85%, in particular of at least 90% and more particularly of at least 95% of the analogue with the PEA-15 protein, and in particular the sequences characteristic of PEA-15 (Renault et al., Biochem. Pharmacol. 2003, 66: 1581), namely the DED domain, and in particular the conserved RXDLF sequence, the NES domain (Nuclear Export Signal), the peptide sequences involved in the interaction of the PEA-15 protein with its various protein partners (for example ERK1/2, Akt, FADD, caspase 8) and the peptide sequences comprising the phosphorylation sites for the protein kinase C(PKC) or for the type II calcium/camoduline-dependant protein kinase, namely respectively LTRIPSAKK (S104) and DIRQPSEEIIK (S116) (S: phosphorylated serine) motifs.

The nature of the modifications which may be introduced into a protein to obtain analogues as defined above and the methods to implement them rely upon the knowledge and the routine practice of one skilled in the art.

According to another of its aspects, the pre-sent invention relates to a screening method of an agent liable to interact with a PEA-15 protein, or an analogue thereof, comprising at least the steps consisting of:
(a) placing at least one PEA-15 protein linked to a support in presence of a compound according to the invention, in conditions suitable for an interaction with said protein,
(b) measuring a first signal S1, characteristic of the assembly obtained in step a),
(c) placing the assembly obtained in step a) in presence of an agent to be screened in conditions suitable for an interaction with said protein,
(d) measuring a second signal S₂, of the same type as S₁, characteristic of the assembly obtained in step c),
(e) comparing S₁ and S₂ in order to draw a conclusion relating to a possible interaction of said PEA-15 protein with the agent to be screened.

According to another of its aspects, the pre-sent invention refers to a method of diagnosis and/or prognosis of a pathological condition liable to involve PEA-15 by detection and, optionally, by quantification of PEA-15 in a biological sample taken from an individual, comprising at least the steps consisting of:
(a) placing at least one PEA-15 protein carrying a fluorescent marker D, or an analogue thereof, in presence of at least one compound, in particular fluorescent, according to the invention in conditions suitable for an interaction with said protein,
(b) A and D being such that they define a fluorescent energy acceptor-donor pair, suitable for the implementation of a fluorescence resonance energy transfer,
(c) measuring a first signal S₁, characteristic of the assembly obtained in step c) by irradiation at a wavelength, which enables the fluorescent energy donor to be excited,
(d) placing the assembly obtained in step a) with a biological sample presumed to include at least one PEA-15 protein, in conditions suitable for the interaction of said PEA-15 protein of the biological sample with said compound according to the invention,
(e) measuring a second signal, S₂ of the same type as S₁, characteristic of the assembly obtained in step c) by irradiation at a wavelength, which enables the fluorescent energy donor to be excited,
(f) comparing S₁ and S₂ in order to draw a conclusion relating to a possible presence of PEA-15 protein in said biological sample, and optionally a conclusion relating to the amount of said protein.

According to another of its aspects, the present invention also refers to an isolated complex comprising at least one PEA-15 protein and at least one compound of the formula according to the invention.

According to another of its aspects, the pre-sent invention also relates to a kit for screening an agent liable to interact with a PEA-15 protein, or an analogue thereof, comprising:
at least one PEA-15 protein carrying a fluorescent marker D or a purification marker, and
at least one compound according to the invention,

optionally, A and D being such that they define a fluorescent energy acceptor-donor pair, suitable for the implementation of a fluorescence resonance energy transfer.

### Compounds

The compounds of the invention are of the following general formula (I):

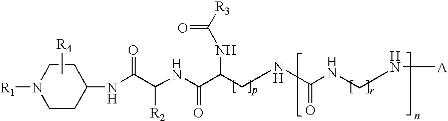

wherein:
n may be equal to 0 or 1,
p may represent an integer varying from 1 to 6, and in particular varying from 2 to 4,
r may represent an integer varying from 1 and 12, in particular varying from 2 to 6, and in particular is equal to 4,
R₁ may represent a hydrogen atom, a saturated or unsaturated, linear or branched C₁-C₂₀ alkyl radical, a saturated or unsaturated C₃-C₁₀ cycloalkyl radical, a C₆-C₁₀ aryl radical, optionally substituted with one or more halogen atom(s), one or more C₁-C₆ alkoxy radical (s), or one or more C₁-C₁₀ alkyl radical (s)
R₂ may represent an amino acid side chain or an amino acid derivative,
—COR₃ may represent an acyl radical, carrier of a basic entity R₃, notably selected from radicals with the following formulas:

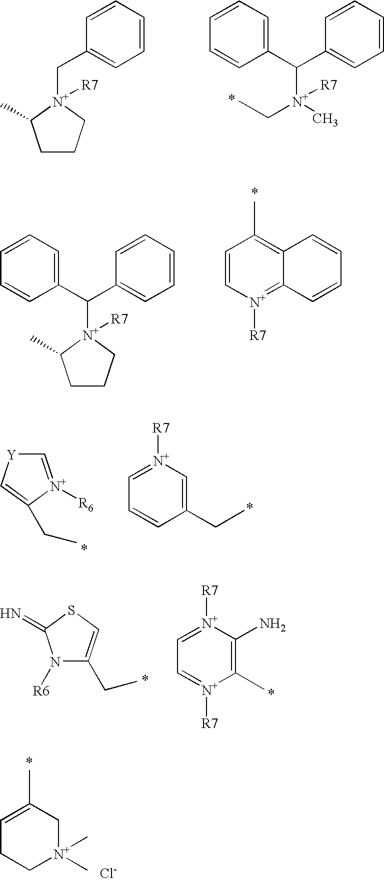

wherein * represents a covalent bond with the acyl radical, Y may represent N or N⁺R₇ and R₆ and R₇ may represent independently from each other, a hydrogen atom, a saturated or unsaturated, linear or branched C₁-C₂₀ alkyl radical, a saturated or unsaturated C₃-C₁₀ cycloalkyl, a C₆-C₁₀ aryl radical, optionally substituted with one or more halogen atom(s), one or more C₁-C₆ alkoxy radical(s), or one or more C₁-C₁₀ alkyl radical(s)
R₄ may represent a hydrogen atom, a saturated or unsaturated, linear or branched C₁-C₁₀ alkyl radical, a saturated or unsaturated C₃-C₁₀ cycloalkyl radical, a C₆-C₁₀ aryl radical, optionally substituted with one or more halogen atom(s), one or more C₃-C₆ alkoxy radicals, or one or more C₁-C₁₀ alkyl radical (s)
A may represent a radical derived from a xanthene residue, in particular of a 9-phenyl xanthene residue, of an acridine residue, in particular of a 9-phenyl acridine residue or of a 4-bora-3a,4a-diaza indacene residue,
and its derivatives.

According to one embodiment, A can represent a fluorescent marker.

According to the invention, “residue” in relation to a given molecule, intends to mean the molecule in the form of a radical.

According to the present invention, “derivative” intends to mean tautomeric forms, stereoisomeric forms, polymorphic forms, pharmaceutically acceptable salts and pharmaceutically acceptable solvates.

According to the present invention, “tautomeric form” intends to mean one of the isomers, the structure of which differs with the position of one atom, generally an hydrogen, and of one or more multiple bonds and which are able to easily and reversibly transform from one into the other.

According to the present invention, “stereoisomeric form” intends to mean isomers of molecules of identical constitution, and which differ only with different arrangements of their atoms in space.

According to the present invention, “pharmaceutically acceptable salts” intends to mean compounds obtained by reaction of a compound of the general formula (I) with a base or an acid.

As example of base suitable for the invention one may mention sodium hydroxide, sodium methoxide, sodium hydride, potassium t-butoxide, calcium hydroxide, magnesium hydroxide, and analogues, and mixtures thereof, in solvents such as THF (tetrahydrofuran), methanol, t-butanol, dioxane, isopropanol, ethanol, analogues, and mixtures thereof.

Organic bases such as lysin, arginine, diethanolamine, choline, tromethamine, guanidine and derivatives thereof may also be used.

As an example of acid additive salts suitable for the invention, one may mention those liable to be prepared by reaction of a compound of the general formula (I) with an acid such as hydrochloric acid, hydrobromic acid, nitric acid, sulphuric acid, phosphoric acid, p-toluenesulfonic acid, methanesulfonic acid, acetic acid, citric acid, maleic acid, salicylic acid, hydroxynapthoic acid, ascorbic acid, palmitic acid, succinic acid, benzoic acid, benzenesulfonic acid, tartaric acid, and analogues, and mixtures thereof, in solvents such as ethyllacetate, ether, alcoholic solvents, acetone, THF, dioxane, analogues and mixtures thereof.

“Polymorphic form” intends to mean compounds obtained by crystallization of a compound of the general formula (I) under various conditions, such as the use of various solvents for example, typically used for crystallization. Crystallization at various temperatures involves, for example, various modes of cooling, very fast to very slow coolings for example, involving heating or fusion steps of compounds followed by gradual or fast cooling. The presence of polymorphic forms can be determined by means of NMR spectroscopy, IR spectroscopy (infrared), DSC (Differentiated Scanning Calorimetry), X-ray diffraction or other similar techniques.

According to the present invention, “radical alkyl” intends to mean a linear or branched, saturated or unsaturated, hydrocarbonated radical, having from 1 to 20 carbon atoms, in particular from 2 to 18 carbon atoms, in particular from 3 to 16 carbon atoms, in particular from 4 to 12 atoms and more particularly from 6 to 10 carbon atoms, liable to be substituted with radicals as defined hereafter.

As example, are included in this definition, radicals such as methyl, ethyl, isopropyl, n-butyl, t-butyl, t-butylmethyl, n-propyl, pentyl, n-hexyl, 2-ethylbutyl, heptyl, octyl, nonyl, or decyl.

According to the present invention, “cycloalkyl radical” intends to mean an alkylene cycle, optionally branched, saturated or unsaturated, having from 3 to 10 carbon atoms, in particular C₄-C₈ and more particularly C₆, such as cyclopropyl, cyclopentyl, cyclohexyl, cyclohexylmethyl, cycloheptyl.

According to the present invention, “aryl radical” intends to mean an aromatic cycle comprising from 1 to 3, possibly fused aromatic ring(s), of 6 to 20 carbon atoms, in particular of 10 to 14 carbon atoms, optionally including one or more heteroatom(s) selected from O, N and S, and if necessary being substituted with radicals as defined above and hereafter.

As example of aryl radicals suitable for the invention, it is possible to mention phenyl radical, benzyl radical, phenethyl radical, naphthyl radical, anthryle radical, and all the aromatic cycles comprising one or more heteroatom(s) selected from O, N and S, such as pyridine, thiophene, pyrrole, furan, quinoline, acridine, xanthene, 4-bora-3a,4a-diaza indacene for example.

According to the present invention, “alkoxy radical” intends to mean an OR-radical wherein the alkyl residue is a linear, branched or cyclic, condensed or not, saturated or unsaturated, hydrocarbonated radical, having from 1 to 20 carbon atoms, in particular from 2 to 18 carbon atoms, in particular from 3 to 16 carbon atoms, in particular from 4 to 12 atoms and more particularly from 6 to 10 carbon atoms.

One may mention, as an example, methoxy, ethoxy, propoxy, butoxy, n-butoxy, isobutoxy, sec-butoxy, n-pentoxy, isopentoxy, sec-pentoxy, t-pentoxy, hexyloxy, methoxyethoxy, methoxypropoxy, ethoxyethoxy, ethoxypropoxy groups and analogues.

According to the present invention, “acyl radical” intends to mean a linear, branched or cyclic condensed or not, saturated or unsaturated hydrocarbonated radical, comprising a C═O moiety and having from 1 to 10 carbon atoms, in particular from 2 to 8 carbon atoms and preferably from 3 to 6 carbon atoms and more particularly having 4 carbon atoms for example, a formyl radical, an acetyl radical, a succinyl radical, a benzoyl radical, a 1-naphthoyle or 2-naphthoyle radical.

The hydrocarbonated chain of said radicals may, if necessary, be interrupted with one or more heteroatoms, for example, selected among O, N and S, to form, for example, an heteroalkyl radical such as an alkylether radical, an alkylester radical or an heterocycle.

According to the present invention, “heterocyclic radical” for example, and in a non-restrictive way, intends to mean a furanyl radical, a thiophenyl radical, a pyrrolyl radical, an oxazolyl radical, a isoxazolyl radical, a thiazolyl radical, a isothiazolyl radical, a imidazolyl radical, a pyrazolyl radical, a furazanyl radical, a pyranyl radical, a pyridinyl radical, a pyridadinyl radical, a pyrimidinyl radical or a pyradinyl radical, a furannyl radical, a quinoleinyl radical.

The above defined radicals may be substituted with one or more halogen atoms if necessary.

According to the present invention, “halogen atom” intends to mean an atom of F, Cl, Br or I. Halogen atoms advantageously implemented in the present invention are fluorine and chlorine.

In particular, the alkylhalogenated radicals may be perfluoroalkyl radicals of the general formula CₙF₂ₙ₊₁ wherein n may vary from 1 to 10, in particular from 2 to 8 and more particularly from 3 to 6.

According to one embodiment, R₁ may notably represent a hydrogen atom, a C₁-C₁₈ alkyl radical, a C₂-C₁₆ alkyl radical, for example a C₆-C₁₀ aryl radical, for example optionally substituted with one or more halogen atom(s).

In particular, R₁ may notably represent a hydrogen atom, a methyl radical, an ethyl radical, an isopropyl radical, a n-propyl radical, a benzyl radical, a phenethyl radical, or a perfluoroalkyl radical of formula CₙF₂ₙ₊₁ in which n may vary from 1 to 10, in particular from 2 to 8 and more particularly from 3 to 6.

In particularly, R₁ may be a methyl radical or a benzyl radical.

According to one embodiment, R₂ may represent an amino acid side chain or an amino acid derivative selected, for example, from the group consisting in alanine, glutamine, leucine, glycine, tryptophan, β-alanine, phenylalanine, 4-chloro-phenylalanine, isonipecotinic acid, 4-aminomethylbenzoic acid, 3-tetrahydroisoquinoleinic acid and free or benzylated histidine.

The amino acid or amino acid derivative may, for example, be selected from the group consisting in:

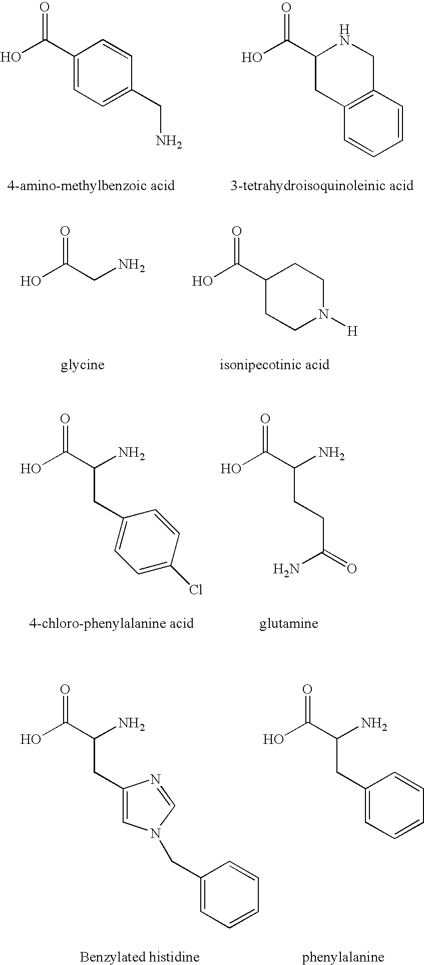

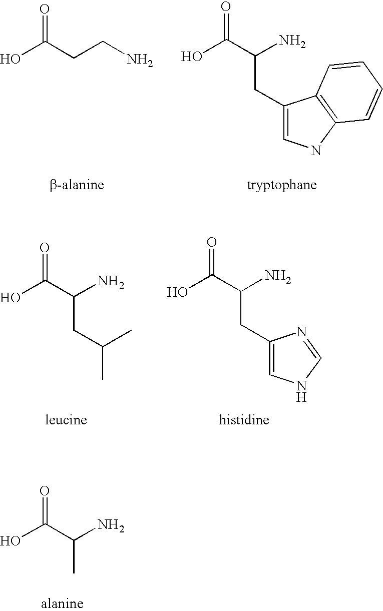

According to one embodiment, R₂ may be of the following formula (VI):

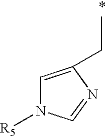

wherein:
* represents a covalent bond with the residue of a compound of the general formula (I), and
R₅ may represent a saturated or unsaturated, linear or branched, C₁-C₂₀ alkyl radical, a saturated or unsaturated, C₃-C₁₀ cycloalkyl radical, a C₆-C₁₀ aryl radical, optionally substituted with one or more halogen atom(s).

According to one embodiment, alkyl or cycloalkyl radicals liable to figure the R₅ radical may also be substituted with the radicals as previously defined or have their hydrocarbon chains interrupted one or more heteroatoms as previously defined.

Particularly, R₅ may represent a hydrogen atom, a C₁-C₁₈ alkyl radical, a C₂-C₁₆ alkyl radical, a C₆-C₁₀ aryl radical, optionally substituted with one or more halogen atom(s).

In particular, R₅ may represent a hydrogen atom, a methyl radical, an ethyl radical, an isopropyl radical, a n-propyl radical, a benzyl radical, a phenethyl radical, a perfluoroalkyl radical of formula CₙF₂ₙ₊₁ wherein n may vary from 1 to 10, in particular from 2 to 8 and more particularly from 3 to 6.

In particular, R₅ may be a methyl radical or a benzyl radical.

R₂ may notably be a histidine or histidine derivative such as a benzylated histidine.

According to one embodiment, —COR₃ may be an acyl radical, notably an acetyl radical, substituted with a basic entity R₃ as previously defined. In particular, this basic entity R₃ may be a radical of the following formula (VII):

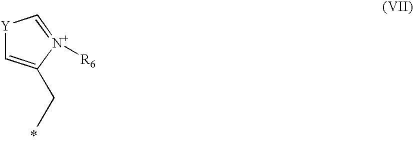

wherein:
* represents a bond with the acyl radical,
Y may represent N or N⁺R₇, and
R₆ and R₇ may represent, independently from each other, a hydrogen atom, a C₁-C₁₈ alkyl radical, a C₂-C₁₆ alkyl radical, a C₆-C₁₀ aryl radical, optionally substituted with one or more halogen atom(s).

In particular, R₆ and R₇ may represent, independently from each other, a hydrogen atom, a methyl radical, an ethyl radical, an isopropyl radical, a n-propyl radical, a benzyl radical, a phenethyl radical, a perfluoroalkyl radical of formula CₙF₂ₙ₊₁ wherein n may vary from 1 to 10, in particular from 2 to 8 and more particularly from 3 to 6.

Particularly, R₆ and R₇ may be, independently from each other, a methyl radical or a benzyl radical.

According to one embodiment, radical A may represent a radical of the general formula (Va):

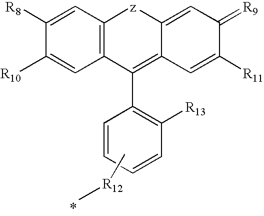

wherein:
* represents a covalent bond with the residue of compound of the formula (I),
Z=O or NH,
R₈═R₉═N(R′)₂, with R′ representing a C₁-C₆, in particular a C₂-C₄ alkyl radical, or R₈═OH and R₉═O,
R₁₀═R₁₁═H or X, with X═F, Cl, Br
or, on the one hand R₈ and R₁₀ and/or on the other hand R₉ and R₁₁ may, respectively, form a 5 or 6-membered heterocycle, condensed with the acridine or xanthenes residue, substituted with one, two, three or even more methyl groups if necessary, and whose heteroatom is placed in a of the acridine or xanthenes residue, and is selected from the group consisting of N and O,
R₁₂=*—NHSO₂— or *—NHCO—, with * representing a covalent bond with the residue of the compound of the formula (I)
—R₁₃═H, HSO₃— or COOH,

In particular, the radical of the formula (Va) may be such as R₈═R₉═NMe₂ or NEt₂,

In particular, the radical A of the formula (Va) may be such, that R₁₂ may be in ortho, meta or paraposition.

In particular, when R₁₂=*—NHSCO₂—, it may be advantageously in ortho-position.

According to one embodiment, A may represent a radical of the general formula (Vb):

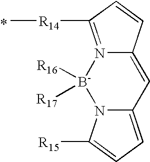

wherein:
* represents a covalent bond with the residue of the compound of the formula (I),
R₁₄ may represent a C₂-C₄ acyl residue,
R₁₅ may represent a C₅-C₇ heterocyclic radical, and
R₁₆═R₃₇═X, with X═F, Cl or Br, and in particular F.

According to one embodiment, the radicals of the formula (Va) and (Vb) may be radicals of fluorescent markers.

As examples of fluorescent markers suitable for the implementation of this invention, it is possible to mention rhodamine and its derivatives such as tetramethylrhodamine, Red-X rhodamine (lissamine), Bodipy and its derivatives, Texas Red® and its derivatives, fluorescein and its derivatives, Alexa® and its derivatives as well as Oregon Green® and its derivatives.

According to one embodiment, radical A may represent a fluorescent marker selected from the group consisting in fluorescent markers of the following formulas:

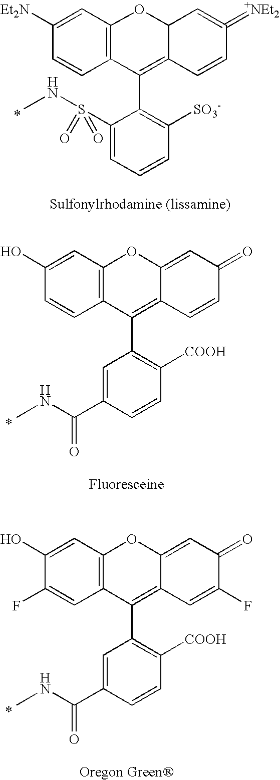

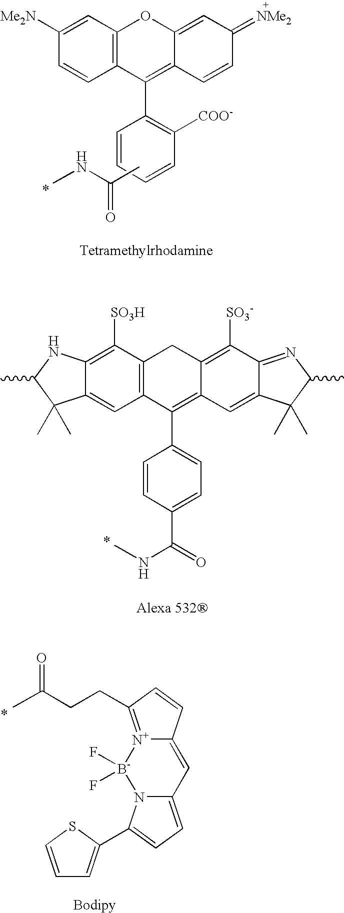

wherein:
* represents a covalent bond with the residue of the compound of the formula (I) according to the invention.

According to one embodiment, the fluorescent marker A may be selected from fluorescent markers derived from rhodamine, such as a sulfonylrhodamine B derivative for example.

In particular, the residue of the compound of the general formula (I) may be bound in ortho-position to the sulfonylrhodamine (lissamine) derivative.

Notably, the radical derived from lissamine may be represented by the radical of the following formula:

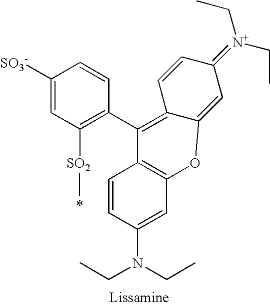

wherein * represents a covalent bond with the residue of the compound of the formula (I) according to the invention.

According to one embodiment, a compound according to the invention may be of the general formula (I) in which n may be equal to 0.

According to one embodiment, a compound according to the invention may be represented, for example, by the following general formula (II):

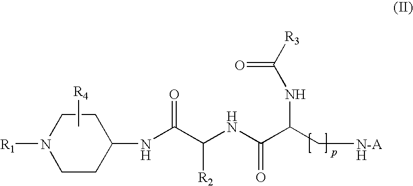

wherein:

R₁, R₂, R₃, R₄, A and p may be as defined previously for example.

According to one embodiment, a compound according to the invention may be of the general formula (I) in which n may be equal to 0, p may be equal to 4, R₁ may represent a methyl radical, R₂ may represent a radical of the following formula:

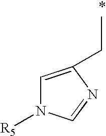

wherein * and R₅ may be as previously defined, and —COR₃ may represent an acyl radical, notably acetyl, substituted with a basic entity R₃, of the following formula:

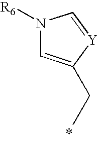

wherein * represents a covalent bond with the acyl radical, and Y and R₆ may be as previously defined, and R₄ may be a hydrogen atom.

According to one embodiment, a compound according to the invention may be of the following general formula (III):

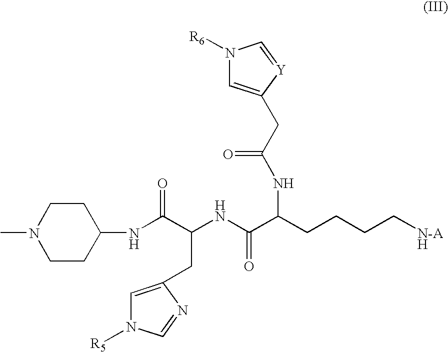

wherein A, R₅, Y and R₆ may be as previously defined.

According to one embodiment, a compound according to the invention may be a compound of the general formula (III) as previously defined, wherein the fluorescent marker A may be, for example, a sulfonylrhodamine radical as defined previously, and notably such as the residue of the compound of the formula (III) is in orthoposition of the sulfonylrhodamine radical, R₅ may be a benzyl radical, Y may be N and R₆ may be a methyl radical.

Advantageously, a compound according to the invention is not a compound of the general formula (I) as previously defined, wherein the fluorescent marker A is a sulfonylrhodamine radical (lissamine) such as the residue of the compound of the formula (I) is in para-position of the sulfonylrhodamine radical.

Advantageously, a compound according to the invention is not a compound of the general formula (III) as previously defined, wherein the fluorescent marker A is a sulfonylrhodamine radical (lissamine) such as the residue of the compound of the formula (III) is in para-position of the sulfonylrhodamine radical, and R₅ is a benzyl radical, Y is N and R₆ is a methyl radical.

According to an alternative embodiment, a compound according to the invention may be represented by the following formula (IV):

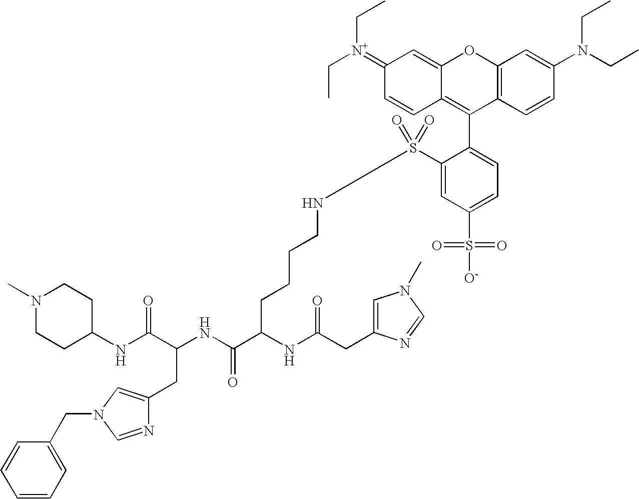

### Synthesis Method

The compounds according to the invention can be obtained either in the form of a library of compounds of varied formulas, or in the form of compounds isolated in pure form or in a mixture of stereo-isomers.

The synthesis method may be carried out on a polystyrene resin of REM type (REgenerated Michael), consisted of a hydroxymethylpoylstyrene resin functionalized by a Michael acceptor acrylic ester.

The REM type resin is particularly suitable for the synthesis of the tertiary amino library, via an initial Michael-type addition of an amina, in order to graft the latter to the support, followed by the synthesis of the molecule on the support, and finally its cut from the support according to an amine quaternization process, then an elimination of HOFFMANN type.

After grafting a secondary amine on the solid support, the other residues may be introduced by means of traditional peptide coupling methods, using the DIC/HOBt activation (1,3-diisopropylcarbodiimide/1-hydroxybenzotriazol).

A radical A, for example, of sulforhodamine type (lissamine, for example), may be grafted directly on an amino function, of the radical ε-NH₂ of a lysin for example or on a spacer grafted on the radical ε-NH₂ of the lysin, such as a diamino-butane spacer, by means of an urethan bond.

The compound(s) may be released from the resin after alkylation of the secondary amine in presence of an alkyl halide such as a methyl iodide, or a benzyl bromide for example, followed by a treatment in the presence of an ion exchange basic resin of the Amberlite IRA-95 type.

According to one embodiment, such a synthesis method according to the invention may be carried out in parallel on plates, for example on 96-well plates, by using FLEXCHEM equipment (Robbins Scientific).

According to one embodiment, a compound according to the invention may be obtained according to a method of preparation on solid support comprising at least the steps consisting of:

a. coupling on a solid support of formula

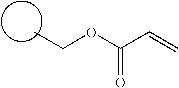

a compound of the formula:

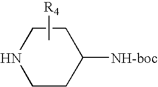

to obtain a compound of the following formula (1):

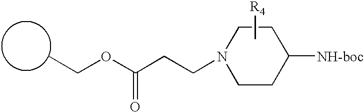

R₄ may be as above defined.

a. deprotecting the compound of the formula (1), then coupling said deprotected compound with the compound of the formula:

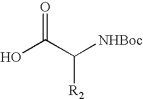

to obtain a compound of the following formula (2):

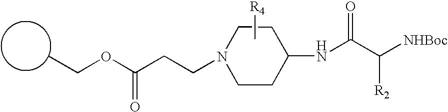

R₂ may be as above defined,

c. deprotecting the compound of the formula (2), then coupling said deprotected compound with the compound of the following formula:

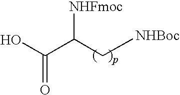

to obtain a compound of the following formula (3):

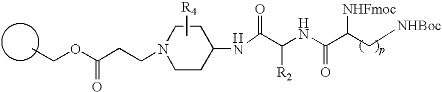

p may be as above defined,

d. deprotecting the compound of the formula (3) from the Fmoc group, then coupling said deprotected compound with a R₃COOH compound to obtain a compound of the following formula (4):

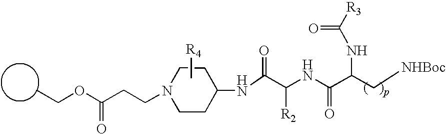

COR₃ being able to be as defined above,

e. deprotecting the compound of the formula (4), to obtain a compound of the following formula (5):

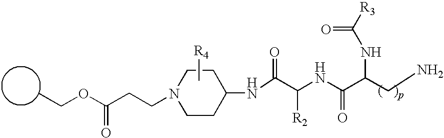

f. optionally, reacting the compound of the formula (5) with the p-nitrophenylchloroformate then with a diamine of formula

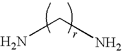

to obtain a compound of the following formula (6):

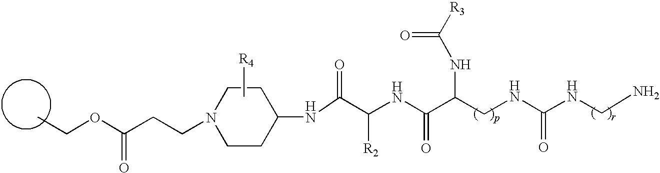

r being able to be as defined above,

g. reacting the compound of the formula (5) or the compound of the formula (6) with an electrophilic tracer, notably with A-Cl, to obtain a compound of the following formula (7):

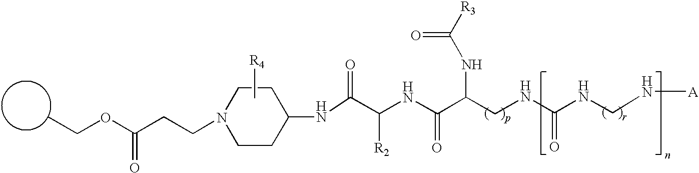

R₂, R₃, A, n, p and r being as defined previously.

h. cleaving the compound of the formula (7) with a compound of the formula R1X, R₁ being as above defined, and X representing a halogen atom, notably I or Br, to obtain a compound of the formula (I) as previously defined.

### Screening and Diagnosis Method

The present invention also relates to a screening method of an agent liable to interact with a PEA-15 protein or an analogue thereof, comprising at least the steps consisting of:
(a) placing at least one PEA-15 protein carrying a fluorescent marker D, or an analogue thereof, in presence of at least one fluorescent compound according to the invention, in conditions suitable for an interaction with said protein, A and D being such that they define a fluorescent energy acceptor-donor pair, suitable for the implementation of a fluorescence resonance energy transfer,
(b) measuring a first signal S₁, characteristic of the assembly obtained in step a), by irradiation at a wavelength, enabling the fluorescent energy donor to be excited,
(c) placing the assembly obtained in step a) in presence of a medium presumed to contain at least one agent to be screened in conditions suitable for an interaction with said protein,
(d) measuring a second signal S₂, of the same type as S₁, characteristic of the assembly obtained in step c) by irradiation at a wavelength enabling the fluorescent energy donor to be excited,
(e) comparing the first and second signals S₁ and S₂ in order to draw a conclusion relating to a possible interaction of said PEA-15 protein with the agent to be screened.

According to the present invention, “A and D being such that they define a fluorescent energy acceptor-donor pair, suitable for the implementation of a fluorescence resonance energy transfer”, intends to mean a pair of fluorescent markers, of which the emission spectrum of one (fluorescent energy donor) covers the whole or a part of the excitation spectrum of the other (fluorescent energy acceptor). In particular, the excitation spectrum of the donor does not cover the excitation spectrum of the acceptor, or a small part thereof, thus avoiding or reducing the occurrence of false positives.

According to one embodiment, the first and second signals may be fluorescence signals of the energy acceptor and/or donor.

In the presence of a compound carrying a fluorescent energy acceptor liable to interact with a compound carrying a fluorescent energy donor, the irradiation of the assembly at a length of the excitation spectrum of the fluorescent energy donor can produce a fluorescence resonance energy transfer (FRET).

According to the invention, “transfer of fluorescent energy” intends to mean a physical process, depending on the distance, by which energy is transmitted, in a non-radiative way, from an excited chromophore, the fluorescent energy donor, to another chromophore, the fluorescent energy acceptor, by dipole-dipole interaction.

The demonstration of such a transfer may be detected by a modulation of the fluorescence signal of the donor and/or fluorescence signal of the acceptor, such as for example a decrease of the amplitude of the fluorescence signal of the fluorescent donor and/or by an increase of the amplitude of the fluorescence signal of the acceptor.

The amplitude variations of the fluorescence signal of the donor may be concomitant with the amplitude variations of the fluorescence signal of the acceptor. Alternatively, one of the fluorescence signals may vary without that a variation in the other fluorescence signal be detected.

The conditions and parameters to adjust in order to carry out a transfer of fluorescent energy rely on the practice of one skilled in the art who can refer to Sekar and Periasamy (J. Cell. Biol., 2003, 160: 629) for example.

According to one embodiment, the comparison of the first and second signals may allow to detect an amplitude modulation of a fluorescence signal.

According to the present invention, “amplitude modulation of a fluorescence signal”, in the context of the fluorescence resonance energy transfer, intends to mean any modulation of the amplitude of the donor fluorescence signal, the amplitude of the excitation spectrum or the amplitude of the donor emission signal as defined previously.

A modulation of these fluorescence signals may mean a possible interaction of the agent to be screened with a PEA-15 protein carrying a fluorescent marker D. Such an interaction may cause the dissociation of a complex PEA-15 protein carrying a fluorescent marker D/compound according to the invention.

According to one embodiment, a screening method according to the invention may further comprise a step consisting of preparing at least one control sample, wherein said medium added in step c) of the method according to the invention previously defined is free of the agent to be screened.

The control sample(s) may be prepared, according to a method according to the invention, simultaneously to or independently of the implementation of such a screening method of an agent liable to interact with PEA-15.

A method according to the invention may comprise a step consisting of comparing a signal S3 measured from a control sample with the signals S1 and S2 such as defined previously, to draw information relating to said agent to be screened.

A difference between the signals thus compared may be informative of the presence, the amount, and/or an interaction with PEA-15, of an agent to be screened in a sample.

According to one embodiment, the fluorescent marker D carried by the PEA-15 protein may be selected, in a non-restrictive way, from the group consisting in a protein, such as a fluorescent protein, a fluorescent marker, for example selected from the group consisting in a fluorescein derivative, a rhodamine derivative, an Alexa 532® derivative, a Bodipy derivative or an Oregon Green® derivative, provided that D and A are as above defined.

According to one embodiment, the fluorescent protein may be selected, in a non-exhaustive way, from the group consisting in the Green Fluorescent Protein, or a fluorescent variant thereof, such as the Yellow Fluorescent Protein (YFP), the Cyan Fluorescent Protein (GFP) or the Red Fluorescent Protein (RFP) or the DS Red, or a variant thereof.

According to one embodiment, the PEA-15 protein carrying a fluorescent marker may in particular be a fusion protein, for example GFP-PEA-15. Such a protein may be obtained by any molecular biology technique known by one skilled in the art, and notably those described in “Molecular Cloning: A Laboratory Manual”, Cold Spring Harbor, Laboratory Cold Spring Harbor, N.Y., 1989, 2d Ed.

The construction and obtaining of an expression vector containing a fusion protein, such as GFP-PEA-15 for example, rely on the knowledge and routine practice of one skilled in the art. The coding sequences for these proteins are available for example in data banks, on the www.ncbi.nlm.nih.gov website or the ca.expasy.org website, and also commercially.

Expression vectors, containing a nucleic acid sequence coding the GFP (or one of its variants) or the DS Red may be commercially available, notably from companies such as Invitrogen or Clontech.

Such vectors may be expressed in any convenient host cell, and the recovery of the fusion protein or if necessary, of the coding nucleic acid for such a protein, such as mRNA or cDNA, may be done by any suitable means known by one skilled in the art.

For example, a GFP-PEA-15 fusion protein has been described by KITSBERG et al. (J. Neurosci., 1999, 19: 8244).

According to one embodiment, a screening method according to the invention may be implemented, for example, by using a compound according to the invention of the formula (IV).

According to one embodiment, the PEA-15 protein carrying a fluorescent marker D may be a GFP-PEA-15 fusion protein.

According to one embodiment, a screening method according to the invention may be carried out ex vivo or in vitro.

For example, a method according to the invention may be carried out ex vivo from a tissue taken from a laboratory animal genetically modified so that its cells express, tissue-specifically or not, a GFP-PEA-15 fusion protein.

A method according to the invention may be carried out in vitro, notably in cellulo from intact cells, or ex cellulo, for example in a cell lysate or after separation of the elements of interest, such as the GFP-PEA-15 fusion protein.

A screening method according to the invention may be carried out in cellulo in cells expressing the fusion protein according to the invention, either after transfection of primary cells of cell lines, by means of an expression vector such as previously defined, or by the culture of cells taken from a laboratory animal genetically modified so as to express a construction as above defined, or by perfusion of primary cells or cell lines, for example by means of a micropipette or any other means known by one skilled in the art, of a fusion protein according to the invention, or a nucleic acid sequence encoding this fusion protein.

According to another of its aspects, the pre-sent invention refers to a screening method of an agent liable to interact with a PEA-15 protein, or an analogue thereof, comprising at least the steps consisting of:
(a) placing at least one PEA-15 protein linked to a support in presence of a compound according to the invention, in conditions suitable for an interaction with said protein,
(b) measuring a first signal S₁ characteristic of the assembly obtained in step a),
(c) placing the assembly obtained in step a) in presence of an agent to be screened, in conditions suitable for an interaction with said protein,
(d) measuring a second signal S₂, of the same type as S1, characteristic of the assembly obtained in step c),
(e) comparing S₁ and S₂ in order to draw a conclusion relating to a possible interaction of said PEA-15 protein with the agent to be screened.

According to one embodiment, the PEA-15 protein may be bound to a support by means of a marker dubbed “purification marker”.

According to the present invention, “purification marker” intends to mean any structure liable to be used for bonding the PEA-15 protein with a support.

A purification marker may be, for example and in a non-restrictive way, a FLAG tag, a polyHistidine tag, or a GST protein (Glutathion S Transferase).

A PEA-15 protein bound to a purification marker such as previously defined may be a fusion protein obtained by any method of molecular biology known by one skilled in the art, notably as indicated above.

For example a GST-PEA-15 fusion protein may be obtained according to the protocol described by Kitsberg et al. (J. Neurosci, 1999, 19: 8244).

According to the considered purification marker, a support suitable for the implementation of the invention may be, for example and in a non-exhaustive way, a surface of a Sepharose bead or a cell culture plate covered with glutathione, such as 96-well plates marketed by SIGMA (ref. P3233), a nickel column, an anti-FLAG antibody bound to a G protein or A protein column, or to the surface of a Sepharose bead or to the bottom of a well of a cell culture plate.

In addition, for an implementation of a method according to the invention, the PEA-15 protein may be bound to the surface of a sensor ship, for implementation in a method of signal detection by surface plasmon resonance, according to means known by one skilled in the art.

According to one embodiment, the compound according to the invention may be fluorescent and the first signal. S₁ and the second signal S₂ may be fluorescence signals.

The measurement of these signals may be obtained by any method of spectrofluorimetry or fluorescence imagery, known by one skilled in the art. The conditions of excitation and recording of the fluorescence emission are to be adapted according to various factors known by one skilled in the art, such as, for example and in a non-exhaustive way, the type of fluorescent marker A, the support on which the PEA-15 protein lies.

A possible interaction of the PEA-15 protein with an agent to be screened causing the displacement of the compound according to the invention bonded beforehand may be detected by a difference in fluorescence intensity between the two signals S₁ and S₂.

According to another embodiment, the first signal S₁ and the second signal S₂ may be signals obtained by surface plasmon resonance, for example by means of a Biacore®-type apparatus, according to protocols known by one skilled in the art.

These signals are independent from the fluorescence or non-fluorescence type of a compound according to the invention.

Thus, in this implementation of the method previously described, the compound according to the invention may be not fluorescent.

The PEA-15 protein may be immobilized on a sensor ship as previously described.

A compound according to the invention may be brought into contact with said protein immobilized to the sensor ship. The interactions between the compound and the protein may be detected by surface plasmon resonance. A possible interaction of the PEA-15 protein with an agent to be screened causing the displacement of the compound according to the invention bonded beforehand may be detected by surface plasmon resonance.

According to another embodiment, the present invention relates to a method of diagnosis and/or prognosis of a pathological condition liable to involve PEA-15 by detection and, possibly, quantification of the PEA-15 protein in at least one biological sample presumed to comprise said protein, comprising at least the steps consisting of:
(a) placing at least one PEA-15 protein carrying a fluorescent marker D, or an analogue thereof, in presence of at least one fluorescent compound according to the invention, in conditions suitable for an interaction with said protein,
(b) A and D being such that they define a fluorescent energy acceptor-donor pair, suitable for the implementation of a fluorescence resonance energy transfer,

(c) measuring a first signal S1 characteristic of the assembly obtained in step a) by irradiation at a wavelength enabling the fluorescent energy donor to be excited,

(d) placing the assembly obtained in step a) in presence of a biological sample presumed to comprise at least one PEA-15 protein, in conditions suitable for the interaction of said PEA-15 protein of the biological sample with said compound according to the invention,

(e) measuring a second signal S₂, of the same type as S₁, characteristic of the assembly obtained in step c) by irradiation at a wavelength enabling the fluorescent energy donor to be excited,

(f) comparing S₁ and S₂ in order to draw a conclusion relating to a possible presence of PEA-15 protein in said biological sample, and possibly a conclusion relating to the amount of said protein.

A comparison of the first and second signals may enable an amplitude modulation of a fluorescence signal to be detected. Such a modulation may be informative of the presence, and possibly, the amount of PEA-15 protein possibly present in the sample.

The determination of the presence and possibly the amount of the PEA-15 protein, possibly compared with reference values obtained, either from a control sample comprising a known amount of this protein, or from a healthy biological sample, possibly in parallel with the previous measure, may be informative of a pathological condition notably involving PEA-15 and/or an evolution of such a condition.

As an example of a pathological condition which may be diagnosed and/or forecasted with a method according to the invention, one may mention cancer, and notably gliomas, ovarian cancers, breast cancers, kidney cancers, melanomas, and also type II diabetes.

A biological sample may be obtained from a biological tissue or a body fluid.

A method according to the invention may comprise a step consisting of comparing a signal S₃ measured from a control sample with the signals S₁ and S₂ as previously defined, to draw information on the presence and, possibly, the amount of PEA-15 in a biological sample.

According to one embodiment, the first and second signals may be compared to one or more fluorescence signals detected from one or more control samples. Such control samples may be obtained by implementing a method according to the invention and by replacing in step c) the biological sample by one or more samples comprising a known amount of PEA-15 protein.

The control sample(s) may be prepared according to a method according to the invention, simultaneously to or independently from the implementation of a method according to the invention, for detection and possibly, the quantification of the PEA-15 protein in a biological sample.

According to one embodiment, it is possible to vary the known amounts of PEA-15 in the control sample(s) or the amount of PEA-15 protein carrying a fluorescent marker D and/or of fluorescent compound according to the invention in such a way as to obtain a reference scale.

A correlation of a fluorescence signal with one of the variable amounts previously defined may be thus accomplished.

Thus, a correlation of a FRET signal may be established with known amounts of PEA-15 protein carrying a fluorescent marker D, of fluorescent compound according to the invention of PEA-15 protein.

According to one embodiment, the PEA-15 protein carrying a fluorescent marker D may notably be as previously defined.

According to one embodiment, the PEA-15 protein carrying a fluorescent marker D may be a fusion protein of GFP-PEA-15 type.

According to one embodiment, the compound according to the invention may be as defined previously, and may notably be of formula (IV) as specified above.

Many variations of a method of diagnosis according to the invention may be considered and combined if necessary with characteristics of a screening method according to the invention.

Thus, according to one embodiment, the invention relates to a method of diagnosis and/or prognosis of a pathological condition which may involve PEA-15, implemented according to the principles indicated above for the screening method, for example, implementing a detection by surface plasmon resonance, wherein the agent to be screened is substituted by the biological sample.

The PEA-15 possibly present in such a sample will be able to bind to the compound according to the invention, and thus to modify the recorded signal.

### Screening or Diagnosis Kit

The present invention also relates to a kit for screening an agent liable to interact with a PEA-15 protein, or of an analogue thereof or for the diagnosis and/or the prognosis of a pathological condition which may involve PEA-15 comprising:
at least one PEA-15 protein carrying a fluorescent marker D or a purification marker, and at least one compound according to the invention,
   optionally, A and D being such that they may define a fluorescent energy acceptor-donor pair, suitable for the implementation of a fluorescence resonance energy transfer.

According to one embodiment, the PEA-15 protein carrying a fluorescent marker D or a purification marker may be as defined previously.

According to one embodiment, when the kit according to the invention is more particularly implemented for the diagnosis and/or prognosis of a pathological condition, it may further comprise at least one unlabelled PEA-15 protein.

According to one embodiment, the fusion protein made up of one PEA-15 protein with a fluorescent protein or the unlabelled PEA-15 protein may be present in a kit according to the invention, in the form of a nucleic acid sequence encoding said proteins, such as cDNA, mRNA, or an expression vector.

### Pharmaceutical Composition

According to one embodiment, the present invention also relates to a compound according to the invention for use as an active agent in a pharmaceutical composition.

According to the present invention, “pharmaceutical composition” means a composition or a substance presented as having curative or preventive properties regarding human or animal diseases, as well as a substance or composition to be used in order to establish a diagnosis and/or prognosis of a pathological or non-pathological condition, or to restore, correct or modify the organic functions of an individual.

The diagnosis and/or prognosis method liable to be implemented through a pharmaceutical composition according to the invention may be carried out in vitro or ex vivo.

A pharmaceutical composition according to the invention may comprise a compound according to the invention of the general formula (I), or one of its derivatives such as a tautomeric form, a stereoisomeric form, a polymorphic form, a pharmaceutically acceptable salt, or a pharmaceutically acceptable solvate, in combination with vehicles, diluents, or excipients ordinarily used in pharmacy.

A pharmaceutical composition according to the invention may be presented in galenic form ordinarily used in the field, such as tablets, capsules, powder, syrup, solution, suspension.

A pharmaceutical composition according to the invention may be presented in galenic form, suitable for administration via other routes, such as oral, nasal, sublingual, topical, ophthalmical, rectal route, etc.

A cosmetic composition according to the invention may also be presented in sterile form, suitable for parenteral administration, such as the subcutaneous, transdermic, intramuscular, intravenous, intra-arterial, intra-cardiac routes, etc.

A pharmaceutical composition according to the invention may also be presented in a freeze-dried form, combined in use with an aqueous solution, sterile or not.

In particular, the aqueous solution may be sterile if the composition according to the invention is to be used for parenteral administration.

The amount of the compound according to the invention present in a pharmaceutical composition is to be adjusted for example according to the administration route, the type of individual to be treated, and the type of pathology to be treated.

The adjustment of the amounts and dosages according to these parameters is known by one skilled in the art.

A composition according to the invention generally comprises a sufficient amount of a compound according to the invention.

“Sufficient amount” intends to mean the amount necessary to obtain a required effect. According to the present invention, such an effect may be the reduction or the treatment of the symptoms presented by an individual for example, possibly having pathology such as cancer or type II diabetes.

The cancer may be glioma, kidney cancer, breast cancer, or melanoma for example.

According to another of its objects, the pre-sent invention relates to the use of a compound according to the invention for the manufacture of a pharmaceutical composition for the treatment of a pathological condition liable to involve PEA-15.

The PEA-15 protein may be involved either by an alteration of its expression, namely an over-expression or a lack of expression for example, or by an alteration of its biological activity, resulting for example in an increase or a reduction of its activity, and possibly being the result either of a mutation (for example substitution, insertion or deletion) in the PEA-15 protein sequence, or of an alteration of the cellular signals modulating the PEA-15 protein biological activity and/or expression.

In relation to a pathological condition which may involve PEA-15, the term “treatment” intends to mean the reduction in the severity of a disease, such as the reduction of the symptoms or the prevention of these symptoms for example.

Thus, and in this latest case, a compound according to the invention may be administered before the development of the pathological condition.

The pathological conditions considered by the present invention may notably be those previously defined, such as cancer, and notably gliomas, kidney cancers, breast cancers, ovarian cancers, melanomas, and also type II diabetes.

“Individual” according to the present invention intends to mean man, non-human primates, as well as laboratory animals such as rodents (mouse, rat, guinea-pig or hamster for example), farm animals, in particular economically interesting animals such as poultry, bovines, sheep, pigs, goats and fish, and in particular those producing products suitable for human consumption such as meat, eggs and milk. This term also describes domestic animals such as cats and dogs.

The present invention also relates to a pharmaceutical composition as previously defined.

According to one embodiment, the present invention also relates to an isolated complex comprising at least one PEA-15 protein carrying a fluorescent marker D or a purification marker and at least one compound of the formula according to the invention, A and D being such that they may define a fluorescent energy acceptor-donor pair, suitable for the implementation of a fluorescence resonance energy transfer.

According to an alternative embodiment, a complex according to the invention may comprise as a PEA-15 protein carrying a fluorescent marker D, a GFP-PEA-15 fusion protein, and as a compound of the formula according to the invention, a compound of the formula (IV) as previously defined.

Many modifications of the invention as above mentioned may be considered by one skilled in the art without leaving the scope of the invention.

Such modifications are covered by the present application.

The invention is illustrated with the following examples, which should not be interpreted as limiting the scope of the present invention.

### FIGURES LEGEND

FIG. 1: represents images obtained by confocal imagery of the location of the intracellular compound of the ERK and PEA-15 proteins before and after treatment with 50 μM of 6D6-1. The treatment of the cells with the 6D6-1 compound results in a relocation of the ERK protein in the core, whereas the PEA-15 protein remains cytoplasmic.

The scale bar corresponds to 40 μm.

FIG. 2: represents the average intensity of the Sepharose bead fluorescence covered with glutathion, carrying a GST-PEA-15 fusion protein, incubated in the presence of 1 and 5 μM of 6D6-1.

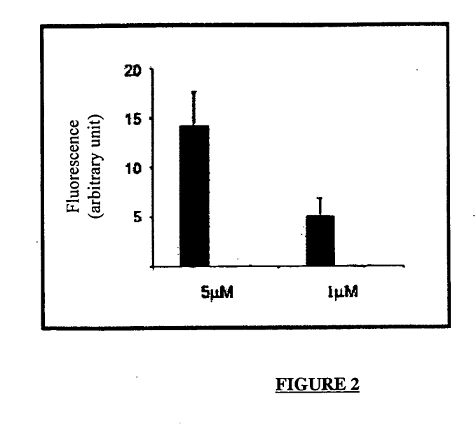

### EXAMPLES

### Example 1

### Synthesis of the 6D6-1 Compound

### Synthesis of (1-Methyl-piperidin-4-yl)-carbamic Acid tert-butyl Ester

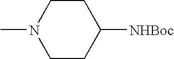

In a reactor, the REM resin (REgenerated Michael, polystyrene resin) (5 g, 4 mmol, 0.8 mmol.g⁻¹ of theoretical load) is inflated in a minimum quality of dimethylformamide (DMF). A solution of tertiobutyloxycarbonylaminopiperidine (8 g, 40 mmol) in DMF (50 ml) is heated to 80° C., then added to the resin in suspension. The mixture is agitated for 16 hours at 80° C., then filtered, and washed three times according to the DMF, CH₂Cl₂ MeOH sequence. In a Supelco syringe, the resin (2.3 g, 1.5 mmol) is expanded in 20 mL of DMF and the methyl iodide (3.81 ml, 61 mmol) is added. The mixture is agitated by rotation for 24 hours, the resin is filtered, washed with 3 sequences of DMF/DCM. In the same conditions, a second step of alkylation with methyl iodide is repeated. The cleavage of piperidin on the resin is accomplished in a balloon with 40 ml of DCM and in the presence of IRA-95 resin (3.16 g, 1.5 mmol). After 24 hours of agitation with a magnetic bar, the resin is filtered, DCM/MeOH washed. The filtrate is collected and dried under reduced pressure. A purification by flash chromatography on silica gel (DCM/MeOH:9/1) results in (1-Methyl-piperidin-4-yl)-carbamic acid tert-butyl ester in the form of white powder.

Yield=100%; ¹H NMR (CDCl₃, 200 MHz): 4.43 (m, 1H), 2.77 (m, 2H), 2.26 (s, 3H), 2.13-1.87 (m, 4H), 1.53-1.46 (m, 2H), 1.42 (s, 9H). ¹³C NMR (CDCl₃, 50 MHz) 155.60, 110.00, 54.86, 46.47, 32.90, 28.80.

### Synthesis of the 1-methyl-piperidin-4-His(Bzl)-NHBoc

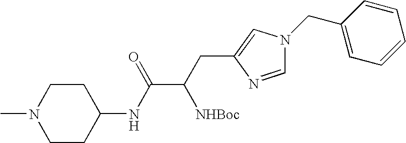

At ambient temperature and under magnetic stirring, the (1-Methyl-piperidin-4-yl)-carbamic acid tert-butyl ester (0.07 g, 0.34 mmol) is treated with a solution of TFA/DCM (1 mL/1 mL) for 90 minutes. The solution is then dry-evaporated under reduced pressure and the obtained product is vacuum-dried for 18 hours. The unprotected N-methyl piperidin is dissolved in 1 ml of DMF and the Boc-His(Bzl)-OH (0.12 g, 0.32 mmol), the benzotriazol-1-yl-oxytrispyrrolidinophosphonium hexafluorophosphate [PyBop] (0.17 g, 0.32 mmol) and the diisopropylethylamine [DIEA] (0.27 ml, 1.6 mmol) are successively added. After a magnetic agitation for 3 hours at ambient temperature, the reaction is dry-evaporated, then purified with HPLC and after freeze-drying, results in a translucent oil.

Yield=100%; tr=15.74 min; X=220 nm; gradient t=0 min: 0% solvent B at t=5 min: 0% solvent B at t=35 min: 100% solvent B. MS (ESI-TOF) m/z (M+H) calculated for [C₂₄H₃₅N₅O₃+H] 442 found 442.

### Synthesis of the Fmoc-Lys(o-Lissamine)-OH

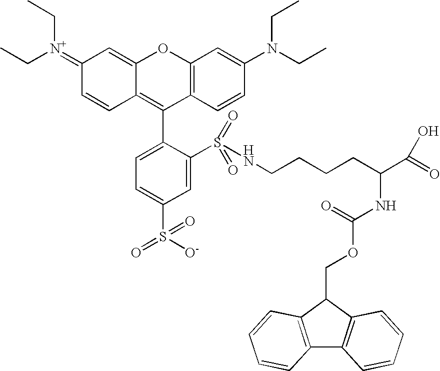

At ambient temperature and under magnetic stirring, the Fmoc-lys(Boc)-OH (0.57 g, 1.23 mmol) is treated with a solution of TFA/DCM (5 mL/5 mL) for 2 hours. The solution is then dry-evaporated under reduced pressure and the obtained product is vacuum-dried for 18 hours. The Fmoc-lys-OH is then dissolved in 17 ml of DCM, then triethylamine [TEA] (1.38 ml, 9.84 mmol) is added to adjust the pH to approximately 8-9, one then observes the formation of a gel which disappears when the lissamine (0.78 g, 1.35 mmol) is added, for 30 minutes at 0° C. Back to ambient temperature, the reaction is left under magnetic stirring for 5 hours. The reaction mixture is then diluted with 50 ml of DCM, washed twice with 10% HCl (10 ml), then the organic phase is dried on sodium sulfate, then dry-evaporated. The purification and the separation of two position isomers are obtained on silica gel (dichloromethane/methanol/acetic acid: 94/5/1) and results in two violet powders.

Yield: 40% para isomer and 5% ortho isomer. MS (ESI-TOF) m/z (M+H) calculated for [C₄₈H₅₂N₄O₁₀S₂+H]909, found 909.

### Synthesis of the 1-methyl-piperidin-4 His(Bzl)-Lys(o-Lissamine)-NHFmoc

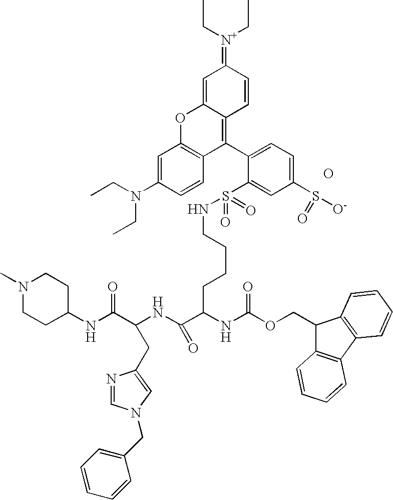

At ambient temperature and under magnetic stirring, the 1-methyl-piperidin-4-His (Bzl)-NHBoc (0.03 g, 0.08 mmol) is treated with a solution of TFA/DCM (1 mL/1 mL) for 90 minutes. The solution is then dry-evaporated under reduced pressure and the product is vacuum-dried for 18 hours. The amine thus obtained is dissolved in 0.5 ml of DMF and the Fmoc-Lys(o-Lissamine)-OH (0.06 g, 0.07 mmol), the PyBop (0.03 g, 0.07 mmol) and the TEA (0.01 ml, 0.07 mmol) are successively added. After a magnetic agitation for 4 hours at ambient temperature, the reaction is dry-evaporated, then purified with HPLC and after freeze-drying, results in a violet powder.

Yield=15%; tr=18.97 min; δ=220 nm; gradient t=0 min: 5% solvent B at t=30 min: 100% solvent B. MS (ESI-TOF) m/z (M+H) calculated for [C₆₇H₇₇N₉O₁₀S₂+H] 1232, found 1232.

### Synthesis of the 1-methyl-piperidin-4-His(Bzl)-Lys(o-Lissamine)-1-methyl-1H-imidazol-4-yl)-acetamide

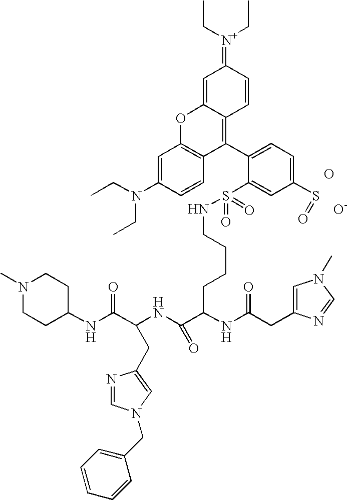

In a first step, the 1-methyl-piperidin-4-His(Bzl)-Lys(o-Lissamine)-NHFmoc (0.01 mg, 0.01 mmol) is treated with 0.12 ml of piperidin in 0.5 ml of DMF for 1 hour at ambient temperature. The solution is then directly injected on semi-preparative HPLC and results in the unprotected product on the final amine with a yield of 40%. The amine (0.004 g, 0.004 mmol) thus obtained is put through a last step of coupling with the 1-methyl-4-imidazoleacetic acid hydrochloride (0.001 g, 0.007 mmol) in the presence of PyBop (0.003 g, 0.007 mmol) and of DIEA (0.005 ml, 0.033 mmol) in 0.3 ml of DMSO. After 90 minutes of stirring at ambient temperature, the solution is directly injected on semi-preparative HPLC and after freeze-drying, results in a violet powder.

Yield 30% tr=17.70 min; X=220 Nm; gradient t=0 min: 5% solvent B at t=35 min: 100% solvent B. MS (ESI-TOF) m/z (M+H) calculated for [C₅₈H₇₃N₁₁O₉S+H] 1132, found 1132.

### Example 2

### Detection of the Interaction of the 6D6-1 Compound with the PEA-15-GFP by FRET

The cell line 3T3 expressing the GFP-PEA-15 (3T3-GFP-PEA-15 cell) was obtained as described by FORMSTECHER et al. (Dev. Cell., 2001, 1:239) by transfection of NIH3T3 cells with a EGFP-PEA-15 plasmide obtained as described by KITSBERG et al. (J. Neurosci., 1999, 19: 8244).

Clones resistant to the neomycin (G418) were selected and cultivated in a DMEM (Roche) medium supplemented with 10% of foetal calf serum, 2 mM of glutamine, penicillin (5 IU/ml) and streptomycin (5 g/ml).

The GFP-PEA-15 protein expression was checked by the measurement of the fluorescence of the living cells and a WESTERN transfer analysis with an anti-GFP antibody (Roche, cat. No. 1,814,460 mixture of two monoclonal antibodies obtained from a mouse (clone 7.1 and clone 13.1)) and an anti-PEA-15 (rabbit polyclonal antibody, Sharif et al., Neuroscience, 126: 263, 2004).

The 3T3-GFP-PEA-15 cells are then cultivated up to confluence in a HAM-F12 medium comprising penicillin (10 000 U/ml)/streptomycin (10 000 μg/ml), 7% of foetal calf serum (BIOWHITTAKER) before the fluorescence resonance energy transfer (FRET) experiences.

A stock solution of the 6D6-1 compound, dissolved in DMSO at a concentration of approximately 20 mm is diluted before use in PBS at a concentration of 10 times the final concentration.

The 6D6-1 compound is tested at 10⁻⁴ M and 10⁻⁵ M.

After adding the 6D6-1 compound, the cells are gently agitated (100 rpm) for 60 minutes before the fluorescence read-out, in order to enable the compounds to diffuse and enter into the cells.

The cells are irradiated at an excitation wavelength of 465 nm.

The GFP protein excited at 465 nm emits a fluorescence signal at 535 nm.

The bond of the 6D6-1 compound to the GFP-PEA-15 protein enables a fluorescence resonance energy transfer (FRET), resulting in the emission of a fluorescence signal at 590 nm.

### Example 3

### Effect of the 6D6-1 Compound on the ERK Location

Primary astrocyte cultures were prepared from cortex and striatum of mouse embryos (day 16) as described by ARAUJO et al. (J. Biol. Chem., 1993, 268: 5911).

The primary astrocyte cultures were maintained for 24 hours in the absence of serum, a condition known to induce a mainly cytoplasmic location of ERK.

The cells were then treated or not with 50 μM of the 6D6-1 compound for 2 hours 15 minutes.

The subcellular location of ERK and PEA-15 was observed by confocal microscopy after labeling the cells with fluorescent antibodies.

The cells were washed twice with PBS (Dulbecco phosphate saline buffer, without CaCl₂ nor MgCl₂, Sigma), and fixed with 4% paraformaldehyde in PBS (pH 7.5), for 15 minutes, then washed twice with PBS comprising 0.1 M of glycine, at ambient temperature.

Then, the cells were incubated for 5 minutes in PBS containing 0.2% of X-100 triton.

The non-specific sites were blocked with PBS containing 10% of normal goat serum (NGS) for one hour at ambient temperature.

Then the cells were incubated overnight at 4° C. with PEA-15 specific antibodies (rabbit polyclonal antibody, Sharif et al., Neuroscience, 2004) or ERK specific antibodies (rabbit polyclonal antibody, Santa Cruz K-23 (ref. Sc-94)), diluted in PBS containing 1.5% of NGS.

After three washings in PBS, the cells are incubated for one hour at ambient temperature with an anti-rabbit antibody labelled by Alexa-488 (Molecular Probes).

The cellular nucleus were labelled with TOPRO² iodide according to manufacturer (HOECHST) specifications.

The lamellae were mounted on glass slides in a FLUOROMOUNT medium (Southern Biotechnology) and examined by confocal microscopy (TCS SP2, LEICA) with the suitable filters.

For the confocal analysis, the excitation wavelengths were 488 nm for Alexa 488 and 633 nm for TOPRO, and the emission wavelengths were 510-525 nm for Alexa-488 and 647 nm for TOPRO.

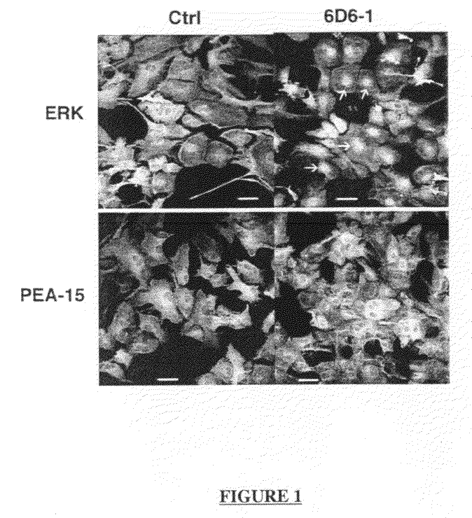

The treatment of the astrocytes with 50 μM of the 6D6-1 compound results in the relocation of ERK in the nucleus, whereas PEA-15 remains cytoplasmic (FIG. 1).

### Example 4

### 6D6-1/PEA-15 Protein Interaction

The GST-PEA-15 fusion protein was obtained according to the protocol described by Kitsberg et al. (J. Neurosci, 1999, 19: 8244).

The GST-PEA-15 fusion proteins were recovered after lysis of bacteria and were incubated together with a Sepharose bead covered with glutathione.

The beads thus obtained are incubated in the presence of 1 or 5 μM of 6D6-1, in a 20 mM Tris-HCl, pH 7.4; 100 mM NaCl; 1 mM MgCl₂; 1% Triton X-100 buffer).

After a series of three washings, the beads are dried on a cellulose membrane and the fluorescence is quantified by means of a phosphoimager (Biorad), by measuring the fluorescence of the lissamine with excitation at 543 nm and measurement of the emission at 590 nm.

The results are the average of three independent experiences.

The results indicate that the average intensity of fluorescence depends upon the concentration of the compound and thus suggests a specific interaction between the 6D6-1 compound and the PEA-15 protein.

1. A compound of the following formula (I): 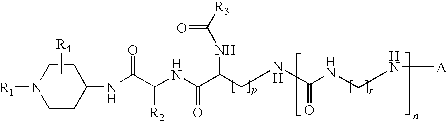
wherein:
n is equal to 0 or 1,
p represents an integer varying from 1 to 6,
r represents an integer varying from 1 to 12,
R₁ represents a hydrogen atom, a saturated or unsaturated, linear or branched, C₁-C₂₀ alkyl radical, a saturated or unsaturated, C₃-C₁₀ cycloalkyl radical, a C₆-C₁₀ aryl radical, optionally substituted with one or more halogen atom(s), one or more C₁-C₆ alkoxy radical(s), or one or more C₁-C₁₀ alkyl radical(s),
R₂ represents a side chain of amino acid or an amino acid derivative,
—COR₃ represents an acyl radical, carrier of a basic entity R₃, selected from following formulas: 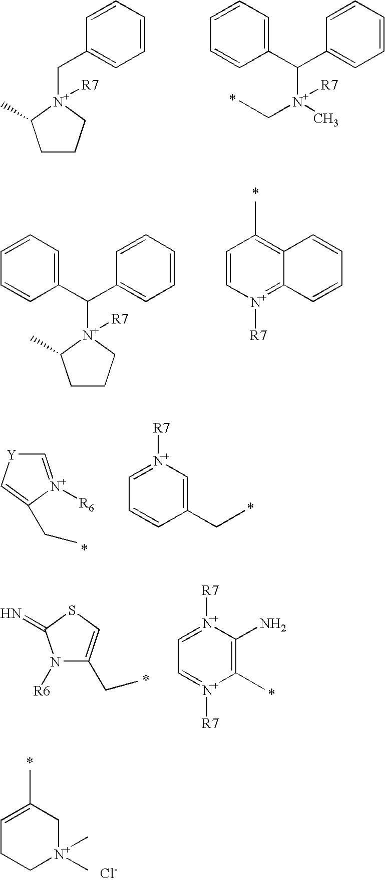
wherein * represents a covalent bond with the acyl radical, Y represents N or N⁺R₇ and R₆ and R₇ represent independently from each other, a hydrogen atom, a saturated or unsaturated, linear or branched, C₁-C₂₀ alkyl radical, a saturated or unsaturated, C₃-C₁₀ cycloalkyl, a C₆-C₁₀ aryl radical, optionally substituted with one or more halogen atom(s), one or more C₁-C₆ alkoxy radical(s), or one or more C₁-C₁₀ alkyl radical(s).
R₄ represents a hydrogen atom, a saturated or unsaturated, linear or branched, C₁-C₁₀ alkyl radical, a saturated or unsaturated, C₃-C₁₀ cycloalkyl radical, a C₆-C₁₀ aryl radical, optionally substituted with one or more halogen atom(s), one or more C₁-C₆ alkoxy radical(s), or one or more C₁-C₁₀ alkyl radical(s),
A represents a radical derived from a xanthene residue, an acridine residue or a 4-bora-3a,4a-diaza indacene residue,
and its derivatives.

2. The compound according to claim 1, wherein A represents a fluorescent marker.

3. The compound according to claim 1, wherein R₁ represents a hydrogen atom, a C₁-C₁₈ alkyl radical, a C₂-C₁₆ alkyl radical, a C₆-C₁₀ aryl radical, optionally substituted with one or more halogen atom(s).

4. The compound according to claim 1, wherein R₁ represents a methyl radical, an ethyl radical, an isopropyl radical, a n-propyl radical, a benzyl radical, a phenethyl radical or a perfluoroalkyl radical of formula CₙF₂ₙ₊₁, wherein n may vary from 1 to 10.

5. The compound according to claim 1, wherein R₂ represents an amino acid side chain or an amino acid derivative selected from the group consisting of alanine, glutamine, leucine, glycine, tryptophan, β-alanine, phenylalanine, 4-chloro-phenylalanine, isonipecotinic acid, 4-aminomethylbenzoic acid, 3-tetrahydroisoquinoleinic acid and free or benzylated histidine.

6. The compound according to claim 1, wherein R₂ is of the following formula (VI): 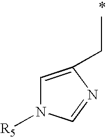
wherein
* represents a covalent bond with the residue of a compound of the general formula (I), and
R₅ represents a saturated or unsaturated, linear or branched, C₁-C₂₀ alkyl radical, a saturated or unsaturated, C₃-C₁₀ cycloalkyl radical, a C₆-C₁₀ aryl radical, optionally substituted with one or more halogen atom(s).

7. The compound according to claim 6, wherein R₅ represents a methyl radical, an ethyl radical, an isopropyl radical, a n-propyl radical, a benzyl radical, a phenethyl radical, a perfluoroalkyl radical of formula CₙF₂ₙ₊₁, wherein n may vary from 1 to 10.

8. The compound according to claim 1, wherein —COR₃ is an acyl radical substituted with a basic entity R₃ of the following formula (VII): 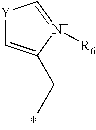
wherein:
* represents a bond with the acyl radical,
Y represents N or N⁺R₇, and
R₆ and R₇ represent, independently from each other, a hydrogen atom, a C₁-C₁₈ alkyl radical, optionally substituted with one or more halogen atom(s).

9. The compound according to claim 8, wherein R₆ and R₇ represent, independently from each other, a hydrogen atom, a methyl radical, an ethyl radical, an isopropyl radical, a n-propyl radical, a benzyl radical, a phenethyl radical, a perfluoroalkyl radical of formula CₙF₂ₙ₊₁, wherein n may vary from 1 to 10.

10. The compound according to claim 1, wherein A represents a radical of the formula (Va): 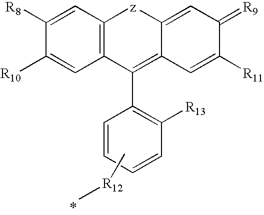
wherein:
* represents a covalent bond with the residue of the compound of the formula (I),
Z=O or NH,
R₈═R₉═N(R′)₂, with R′ representing a C₁-C₆, in particular a C₂-C₄ alkyl radical or R₈═OH and R₉═O,
R₁₀═R₁₁═H or X, with X═F, Cl, Br,
or, on the one hand R₈ and R₁₀ and/or on the other hand R₉ and R₁₁, respectively, form a 5 or 6 membered heterocycle, condensed with the acridine or xanthene residue, substituted with one, two, three methyl groups if necessary, and whose heteroatom is placed in α of the acridine or xanthene residue, and is selected from N or O,
R₁₂=*—NHSO₂— or *—NHCO—, with * representing a covalent bond with the residue of the compound of the formula (I)
R₁₃═H, HSO₃— or COOH,
or a radical of the formula (Vb): 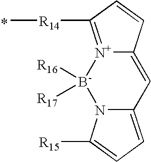
wherein:
* represents a covalent bond with the residue of the compound of the formula (I),
R₁₄ represents a C₂-C₄ acyl residue,
R₁₅ represents a C₅-C₇ heterocyclic radical, and
R₁₆═R₁₇═X, with X═F, Cl or Br.

11. The compound according to claim 10, wherein R₁₂ is in ortho-position.

12. The compound according to claim 2, wherein the radical A represents a fluorescent marker selected from the group consisting of Bodipy and its derivatives, rhodamine and its derivatives, sulforhodamine 101 sulfonyl chloride and its derivatives, fluorescein and its derivatives, Alexa® and its derivatives and 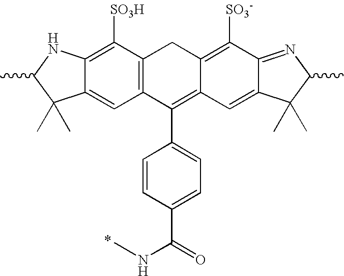
and its derivatives.

13. The compound according to claim 12, wherein the fluorescent marker A is selected from the group consisting of fluorescent markers with the following formulas: 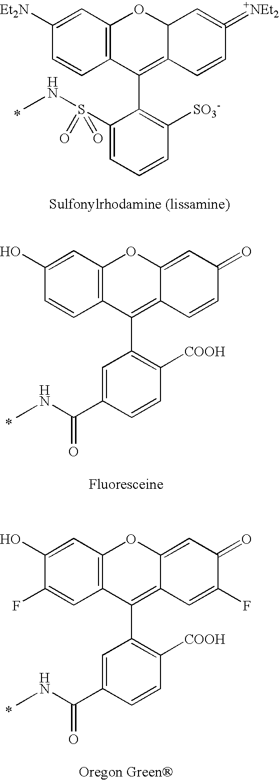 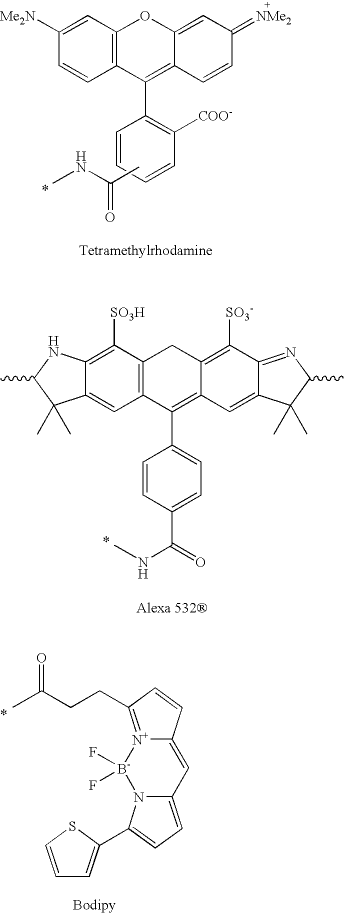
wherein * represents a covalent bond with the residue of the compound of the formula (I).

14. The compound according to claim 1, wherein n is equal to 0.

15. The compound according to claim 1, represented by the following formula (II):

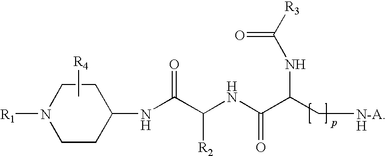

16. The compound according to claim 9, represented by the following formula (III): 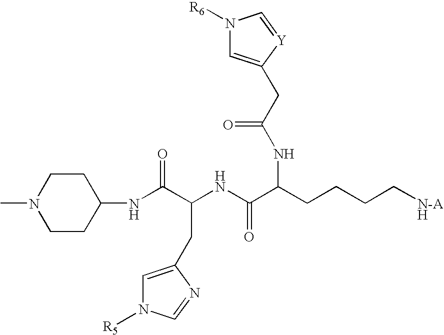
wherein R₅ represents a saturated or unsaturated, linear or branched, C₁-C₂₀ alkyl radical, a saturated or unsaturated C₁-C₁₀ cycloalkyl radical, a C₆-C₁₀ aryl radical, optionally substituted with one or more halogen atom(s).

17. The compound according to the claim 16, represented by the following formula (IV):

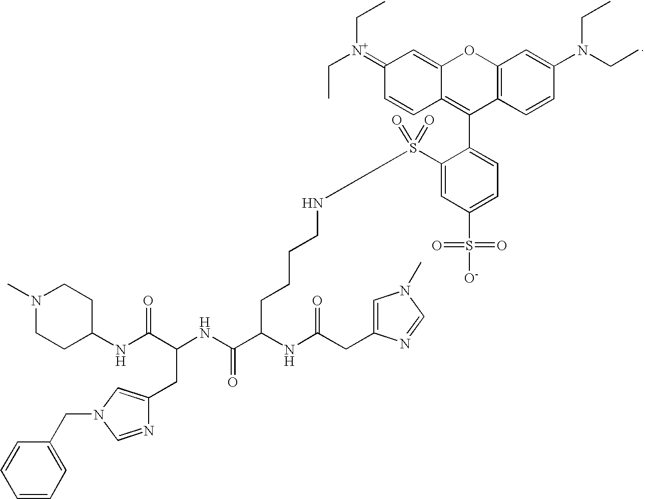

18. A screening method of an agent liable to interact with a PEA-15 protein or an analogue thereof, comprising at least the steps of:
a) placing at least one PEA-15 protein linked to a support in presence of a compound according to claim 1, in conditions suitable for an interaction with said protein to form an assembly,
b) measuring a first signal S₁, characteristic of the assembly obtained in step a),
c) placing the assembly obtained in step a) in presence of an agent to be screened in conditions suitable for an interaction with said protein to form an assembly,
d) measuring a second signal S₂, of the same type as S₁, characteristic of the assembly obtained in step c),
e) comparing S₁ and S₂ in order to draw a conclusion relating to a possible interaction of said PEA-15 protein with the agent to be screened.

19. A screening method of an agent liable to interact with a PEA-15 protein or an analogue thereof, comprising at least the steps consisting of:
a) placing at least one PEA-15 protein carrying a fluorescent marker D, or an analogue thereof, in presence of at least one compound according to claim 2, in conditions suitable for an interaction with said protein to form an assembly,
A and D being such that they define a fluorescent energy acceptor-donor pair, suitable for the implementation of a fluorescence resonance energy transfer,
b) measuring a first signal S₁, characteristic of the assembly obtained in step a) by irradiation at a wavelength, enabling the fluorescent energy donor to be excited,
c) placing the assembly obtained in step a) in presence of a medium presumed to contain at least one agent to be screened in conditions suitable for an interaction with said protein to form an assembly,
d) measuring a second signal S₂, of the same type as S₁, characteristic of the assembly obtained in step c) by irradiation at a wavelength, enabling the fluorescent energy donor to be excited,
e) comparing the first and second signals S₁ and S₂ in order to draw a conclusion relating to a possible interaction of said PEA-15 protein with the agent to be screened.

20. The method according to claim 19, wherein the fluorescent marker D is selected from the group consisting of a fluorescent protein, and a fluorescent marker selected from the group consisting of a fluorescein derivative, a rhodamine derivative, a derivative of 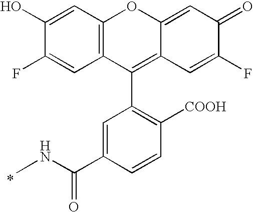
a Bodipy derivative or a derivative of

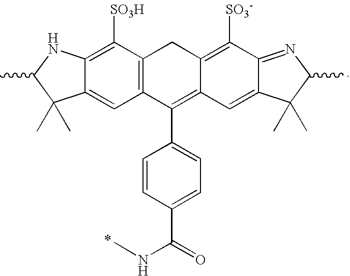

21. The method according to claim 20, wherein the fluorescent protein is selected from the group consisting of Green Fluorescent Protein, or one of its fluorescent variants, or DS Red, or one of its variants.

22. The method according to claim 20, wherein the PEA-15 protein carrying a fluorescent marker D is a GFP-PEA-15 fusion protein.

23. The method according to claim 19, carried out in cellulo.

24. The method according to claim 23 carried out in cells expressing a GFP-PEA-15 fusion protein.

25. A method of diagnosis and/or prognosis of a pathological condition liable to involve PEA-15 by detection and, optionally, by quantification of the PEA-15 protein in at least one biological sample presumed to include said protein, comprising at least the steps of:
a) placing at least one PEA-15 protein carrying a fluorescent marker D, or an analogue thereof, in presence of at least one compound according to claim 2, in conditions suitable for an interaction with said protein to form an assembly,
A and D being such that they define a fluorescent energy acceptor-donor pair, suitable for the implementation of a fluorescence resonance energy transfer,
b) measuring a first signal S₁, characteristic of the assembly obtained in step a) by irradiation at a wavelength, which enables the fluorescent energy donor to be excited,
c) placing the assembly obtained in step a) in presence of a biological sample presumed to include at least one PEA-15 protein, in conditions suitable for the interaction of said PEA-15 protein of the biological sample with said compound to form an assembly
d) measuring a second signal, S₂ of the same type as S₁, characteristic of the assembly obtained in step c) by irradiation at a wavelength which enables the fluorescent energy donor to be excited,
e) comparing S₁ and S₂ in order to draw a conclusion relating to a possible presence of the PEA-15 protein in said biological sample, and optionally a conclusion relating to the amount of said protein.

26. The method according to claim 25, wherein the fluorescent marker D is selected from the group consisting of a fluorescent protein and a fluorescent marker selected from the group consisting of a fluorescein derivative, a rhodamine derivative, a derivative of 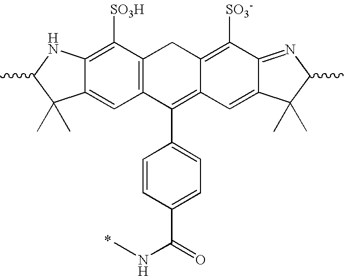
a Bodipy derivative or a derivative of

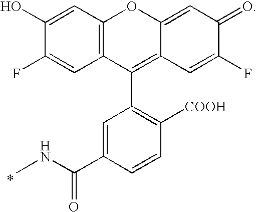

27. An isolated complex including at least one PEA-15 protein and at least one compound according to claim 1.

28. A kit for screening an agent liable to interact with a PEA-15 protein, or an analogue thereof, or for the diagnosis and/or prognosis of a pathological condition liable to involve PEA-15, comprising:
at least one PEA-15 protein carrying a fluorescent marker D or a purification marker, and
at least one compound according to claim 1,
optionally, A and D being such that they define a fluorescent energy acceptor-donor pair, suitable for implementation of a fluorescence resonance energy transfer.

29. (canceled)

30. A pharmaceutical composition comprising at least one compound according to claim 1.

31. A method for treatment of a pathological condition involving PEA-15 comprising administering to a patient in need of said treatment the compound according to claim 1.

32. The method according to claim 21, wherein the fluorescent variant of the Green Fluorescent Protein is selected from the group consisting of Yellow Fluorescent Protein (YFP), Cyan Fluorescent Protein (CFP) and Red Fluorescent Protein (RFP).
